SEQUENCE LISTING (4 sequences)

SEQ ID NO: 1 - protein, 106 aa, synthetic construct
DIVLTQSPSSLSASLGERVSLTCRASQDIHGYLNLFQQKPGETKHLIYETSNLDSGVPKRFSGSRSGSDYSLIIGSLESEDFADYYCLQYASSPRTFGGGTKLEIK

SEQ ID NO: 2 - protein, 110 aa, synthetic construct
DIVMTQSPASLAVSLGQRATISCRASESVDNYGISFMNWFQQKPGQPKLLIYAASNQGSGVPARFSGSGSGTDFSLNIHPMEEDDTAMYFCQQSKEVPWTFGGGTKLEMK

SEQ ID NO: 3 - protein, 116 aa, synthetic construct
EVLLQQSGAELARPGASVKLSCKTSGYTFTSYGINWVKQRTGQGLEWIGEILPGSGSTNYNEKFKGKATFTADTSSNTAYMQLSSLTTEDSAIYYCARDGYYGDYWGQGTTLTVSS

SEQ ID NO: 4 - protein, 118 aa, synthetic construct
EVQLQQSGAELVKPGASVKLSCKASGYTFTEYTIHWVKQRSGQGLEWIGETLPGRGSTNYNEKFKGKATFTTDTSSNTAYMQLSSLTPEDSAIYYCARGTGTKNFDCWGQGTTLTVSS

(19) United States
(12) Patent Application Publication
(10) Pub. No.: US 2026/0102768 A1
(43) Pub. Date: Apr. 16, 2026

(54) LAB-ON-A-CHIP FOR CHEMILUMINESCENCE-BASED MICROCHANNEL LATERAL FLOW ASSAY

(71) Applicant: University of Cincinnati, Cincinnati, OH (US)

(72) Inventors: Chong H. Ahn, Cincinnati, OH (US); Heeyeong Jang, Cincinnati, OH (US); Supreeth Setty, Cincinnati, OH (US)

(73) Assignee: University of Cincinnati, Cincinnati, OH (US)

(21) Appl. No.: 19/355,438

(22) Filed: Oct. 10, 2025

Related U.S. Application Data
(60) Provisional application No. 63/706,414, filed on Oct. 11, 2024.

(51) Int. Cl. B01L 3/00; G01N 33/543; G01N 33/569

(52) U.S. Cl. CPC B01L 3/502715; B01L 3/50273; G01N 33/54388; G01N 33/54393; G01N 33/56983; B01L 2200/10; B01L 2200/16; B01L 2300/0883; B01L 2300/165; B01L 2400/0406

(57) ABSTRACT

Disclosed herein is a microchannel lateral flow assay lab-on-a-chip device for point-of-care testing for detecting a target in a sample. The chip has a surface comprising a sample loading channel configured to receive the sample, a detection antibody conjugate channel configured to receive a first portion of the sample, a chemiluminescent substrate channel configured to receive a second portion of the sample, one or more reaction channels, wherein at least one of the reaction channels generates a signal of immunoassay, a first delay line between the chemiluminescent substrate channel and the one or more reaction channels, a capillary pump, and a film over the surface of the chip. Also disclosed are methods for detecting a target in a sample, methods for making microchannel lateral flow assay lab-on-a-chip devices, and systems for detecting a target in a sample.

### CROSS-REFERENCE TO RELATED APPLICATIONS

The present application claims priority to U.S. Provisional Application No. 63/706,414, filed Oct. 11, 2024, the entirety of which is incorporated herein by reference.

### SEQUENCE LISTING

The instant application contains a Sequence Listing which has been submitted electronically in XML format and is hereby incorporated by reference in its entirety. Said XML copy, created on Oct. 9, 2025, is named “CIN0433PA.xml” and is 4,937 bytes in size.

### TECHNICAL FIELD

This disclosure relates to the field of point-of-care testing (POCT). Specifically, this disclosure relates to lab-on-a-chip platforms for high sensitivity POCT.

### BACKGROUND

The well-known criteria for point-of-care-test (POCT), known as ASSURED which stands for Affordable, Sensitive, Specific, User-friendly, Rapid & robust, Equipment-free, and Delivered, were established by the World Health Organization (WHO) in 2004. To meet the criteria, over the past two decades, numerous efforts were made for the development of biochemical analysis laboratories or systems in micro scale which included lab-on-a-chip (LOC) and micro total analysis systems (μTAS). When it comes to POCT, there's always a trade-off with one or the other ASSURED criterion compared to the gold standard conventional clinical laboratory test. Among these criteria, sensitivity should be one of the most important, as it defines the proportion of true positive tests out of all patients with a condition. Another way to define this criterion is the position of biomarker's limit of detection (LoD) with respect to the Upper Reference Limit (URL) of healthy population and it is commonly known as analytical sensitivity. POCTs benefit from microfluidic technologies, which allow a rapid response and high-sensitive reaction, requiring only small volumes of reagents or samples. POCT devices popularly known as rapid diagnostic tests (RDTs), based on the immunochromatographic technique using a cellular membrane to immobilize the sensing chemistry and transport samples and reagents, are often available without a doctor's prescription as an Over-the Counter (OTC) device. In RDTs, analytical sensitivity is less critical, since such tests are primarily used for screening purposes only and provide qualitative results.

Commonly employed optical detection methods for Enzyme Linked Immunosorbent Assay (ELISA) include absorbance, fluorescence, and chemiluminescence (CL). In recent years, CL-based ELISA tests have gained attention due to their high-sensitivity and simplicity of optical detection, but they require the use of fresh, liquid CL substrate, making such devices pseudo-POCTs. To make them suitable for POCT, it is necessary to store reagents on the chip in a dried format.

One of the most challenging issues in the realization of CL-based microchannel lateral flow assay (CL-mLFA) in POCT platform is sequentially and timely delivering the reagents stored on the lab chip to the reaction spirals. This requires multiple flow paths and controls to ensure an effective reconstitution of dried-reagent complexes, uniform washing, and enzyme-catalyzed signal generation before the loaded sample reaches the reaction spirals. Prior attempts have required multiple additions of ELISA reagents in liquid format at the inlet port, which is not suitable for a sample-to-answer method of POCT. Other attempts that utilized lyophilized reagents involved multiple manual steps, such as venting or operating valves, that are also not suitable for a sample-to-answer method of POCT.

Therefore, a need exists for the development of lab-on-a-chip (LOC) platforms with new assay and detection protocols for high-sensitivity point-of-care testing (POCT) applications.

### SUMMARY

Accordingly, provided herein is a novel lab-on-a-chip device for carrying out chemiluminescence-based microchannel lateral flow assays.

According to one or more embodiments, disclosed herein is a microchannel lateral flow assay lab-on-a-chip device for point-of-care testing for detecting a target in a sample. The chip has a surface comprising a sample loading channel configured to receive the sample, a detection antibody conjugate channel comprising a dried or lyophilized detection antibody conjugated with an enzyme, the detection antibody conjugate channel configured to receive a first portion of the sample, a chemiluminescent substrate channel comprising a dried or lyophilized chemiluminescent substrate, wherein the chemiluminescent substrate channel is configured to receive a second portion of the sample, one or more reaction channels connected to the detection antibody conjugate channel and the chemiluminescent substrate channel, wherein at least one of the reaction channels generates a signal of immunoassay, and the at least one reaction channel that generates a signal of immunoassay comprises immobilized capture antibodies, a first delay line between the chemiluminescent substrate channel and the one or more reaction channels, the first delay line comprising a coating, a capillary pump, and a film over the surface of the chip.

According to one or more embodiments, also disclosed herein is a method for detecting a target in a sample using the microchannel lateral flow assay lab-on-a-chip device according to one or more embodiments of the present disclosure. The method may comprise loading the sample into the sample loading channel, allowing the first portion of the sample to flow through the detection antibody conjugate channel and the one or more reaction channels, wherein the first portion of the sample exits the one or more reaction channels before the second sample enters the one or more reaction channels, and identifying any immunoassay signal.

According to one or more embodiments, also disclosed herein is a method for making the microchannel lateral flow assay lab-on-a-chip device according to one or more embodiments of the present disclosure. The method may comprise forming the chip via injection molding, forming an elastomer dam structured and arranged to prevent liquid flow from the detection antibody conjugate channel to adjacent channels, adding a liquid detection antibody conjugated with an enzyme to the detection antibody conjugate channel, drying or lyophilizing the liquid detection antibody conjugated with an enzyme to form the dried or lyophilized detection antibody conjugated with an enzyme, and removing the elastomer dam, forming an elastomer dam structured and arranged to prevent liquid flow from the chemiluminescent substrate channel to adjacent channels, adding a liquid detection chemiluminescent substrate to the chemiluminescent substrate channel, drying or lyophilizing the liquid chemiluminescent substrate to form the dried or lyophilized chemiluminescent substrate, and removing the elastomer dam, depositing the coating to the first delay line, and depositing the film over the surface of the chip.

According to one or more embodiments, also disclosed herein is a system for detecting a target in a sample. The system may comprise the microchannel lateral flow assay lab-on-a-chip device according to one or more embodiments disclosed herein. The system may further comprise an electronic reader structured and arranged to receive and read the microchannel lateral flow assay lab-on-a-chip. The electronic reader may comprise an optical detection unit structured and arranged to read the optical signal of the chemiluminescent signal and produce a current corresponding to such reading, an electronic control unit comprising a trans-impedance amplifier circuit structured and arranged to convert the current to a signal voltage and amplify the signal voltage based on feedback resistance using a potentiometer, an analog-digital converter structured and arranged to convert the signal voltage to a digital signal that is transferred to a microcontroller via a serial peripheral interface, and a computer processing unit structured and arranged to receive the digital signal from the microcontroller. The electronic reader may further comprise a user interface structured and arranged to provide a result derived from the digital signal.

Additional features and advantages of the articles described herein will be set forth in the detailed description which follows, and in part will be readily apparent to those skilled in the art from that description or recognized by practicing the embodiments described herein, including the detailed description which follows, the claims, as well as the appended drawings.

### BRIEF DESCRIPTION OF THE DRAWINGS

It is to be understood that both the foregoing general description and the following detailed description describe various embodiments and are intended to provide an overview or framework for understanding the nature and character of the claimed subject matter. The accompanying drawings are included to provide a further understanding of the various embodiments, and are incorporated into and constitute a part of this specification. The drawings illustrate the various embodiments described herein, and together with the description serve to explain the principles and operations of the claimed subject matter.

FIG. 1 is a simplified block diagram showing the sequential dual flow, according to one or more embodiments;

FIG. 2 is a schematic illustrating a dual flow LOC for chemiluminescence-based microchannel lateral flow assay (CL-mLFA), according to one or more embodiments;

FIG. 3 is a series of panels (a)-(e) illustrating reagent flow within the LOC, including the autonomous two-path flow sequence after sample loading, according to one or more embodiments;

FIG. 4 is an assay protocol schematic for CL-mLFA showing (a) the negative, positive and test spiral reaction channels after preparation of assay, (b) the spiral reaction channels filled with reactive molecules after the first flow path detection antibody reconstitution, (c) unbonded molecules washed away by remaining sample from the first flow path, and (d) reconstituted CL substrate in the second flow path reacting with HRP in the spiral reaction channels to generate a chemiluminescence signal, each according to one or more embodiments;

FIG. 5 is a schematic for the design, microfabrication, and assay preparation of the mLFA-LOC, according to one or more embodiments, including: (a) a 3D CAD design of the master mold along with the toolpath for machining, the 76.2 mm aluminium micromachined master mold and the 76.3 mm aluminium master mold inspected using contour microscope Bruker GT-X to measure the machined dimensions (Table I), (b) an illustration of the injection molding process using polystyrene in BOY22, (c) a chip released after injection molding, followed by surface modifications, lyophilization and sealed with a pressure sensitive adhesive tape, and (d) a photograph of a fully prepared polymer LOC to perform the test, according to one or more embodiments;

FIG. 6 is a flow chart for the lyophilization of a one-component CL substrate, according to one or more embodiments;

FIG. 7 is a bar graph showing the first delay line delay flow time relative to the water contact angle, according to one or more embodiments;

FIG. 8A is a graphical representation of a comparison of chemiluminescent intensity of one component CL intensity before (original) and after lyophilization (processed), according to one or more embodiments;

FIG. 8B is UV-Vis absorption spectra of one component CL substrate before (original) and after lyophilization (processed), according to one or more embodiments;

FIG. 9 is VL and VH amino acid sequence of scFv2 and scFv4 antibodies, where panels (a) and (b) show the amino acid sequences for scFv2, and panels (c) and (d) show the amino acid sequences for scFv4, respectively, according to one or more embodiments;

FIG. 10A is a block diagram of the opto-electronic system categorized into User interface, Electronic control and Optical detection, according to one or more embodiments;

FIG. 10B is a block diagram illustrating the flow of opto-electronic signals in the portable reader for quantifying the CL signal intensity, according to one or more embodiments;

FIG. 10C is a block diagram illustrating the components of the portable reader, according to one or more embodiments;

FIG. 11 is a custom designed portable reader with an enclosure and a slot to insert the fully assembled mLFA-LOC device cartridge providing alignment with the photodiodes and prevents cross-talk, according to one or more embodiments;

FIG. 12 is a schematic illustration of the overall approach for sequential dual-flow LOC with on-chip lyophilized one-component chemiluminescence substrate for high-sensitive mLFA, detected using a custom designed portable reader, according to one or more embodiments;

FIG. 13 is a graph of voltage compared to chemiluminescence intensity generated by a portable reader showing a trend for CL signal obtained from various concentrations of DAb-HRP in reaction zones of mLFA for a gain resistance of 100 KΩ, compared with BioTek reader, according to one or more embodiments;

FIG. 14 is a graph of CL intensity on the Optimiser microplate for N-protein extracted from pseudovirus, detected using the custom designed scFv antibody pair, according to one or more embodiments, and compared with the commercially available IgG antibody pair (Sino Bio);

FIG. 15 is a graph of CL intensity on Optimiser microplate for N-protein in a spiked sample;

FIG. 16 is a bar graph of CL signal intensity obtained from a test reaction channel compared to the number of wash steps performed for that reaction zone;

FIG. 17 is a graph for the assay with N-protein extracted from pseudovirus on mLFA, quantified using a developed portable reader, according to one or more embodiments, and compared to a commercial reader (BioTek); and

FIG. 18 is a graph obtained for N-protein assay with spiked sample on mLFA, quantified using the portable reader, according to one or more embodiments, and compared with BioTek reader.

Reference will now be made in greater detail to various embodiments of the present disclosure, some embodiments of which are illustrated in the accompanying drawings. Whenever possible, the same reference numerals will be used throughout the drawings to refer to the same or similar parts.

### DETAILED DESCRIPTION

Described herein are microchannel lateral flow assay lab-on-a-chip devices for point-of-care testing for detecting a target in a sample. In embodiments described herein, the microchannel lateral flow assay lab-on-a-chip device 10 includes multiple flow paths (300A, 400A) where at least one flow path 400A includes a chemiluminescent substrate channel 400, another flow path 300A includes a detection antibody conjugate channel, and the flow paths merge at one or more reaction channels 500 to generate a signal of immunoassay. The present disclosure is also directed to cartridges 1000 that house the microchannel lateral flow assay lab-on-a-chip device 10, methods of detecting a target biomolecule using the microchannel lateral flow assay lab-on-a-chip device 10, methods of making the microchannel lateral flow assay lab-on-a-chip device 10, and a system 2000 for detecting a target in a sample. These embodiments are described in detail herein.

As discussed above, a disadvantage of prior microchannel lateral flow assay lab-on-a-chip devices is the complicated tasks of handling multiple reagents manually in liquid form and a lack of automation that requires other manual steps to be performed by a user, such as various vent openings or operating of air pumps.

The present disclosure aims to solve this problem by including structural features in the microchannel lateral flow assay lab-on-a-chip device that achieve controllable sequential dual-flow of the sample.

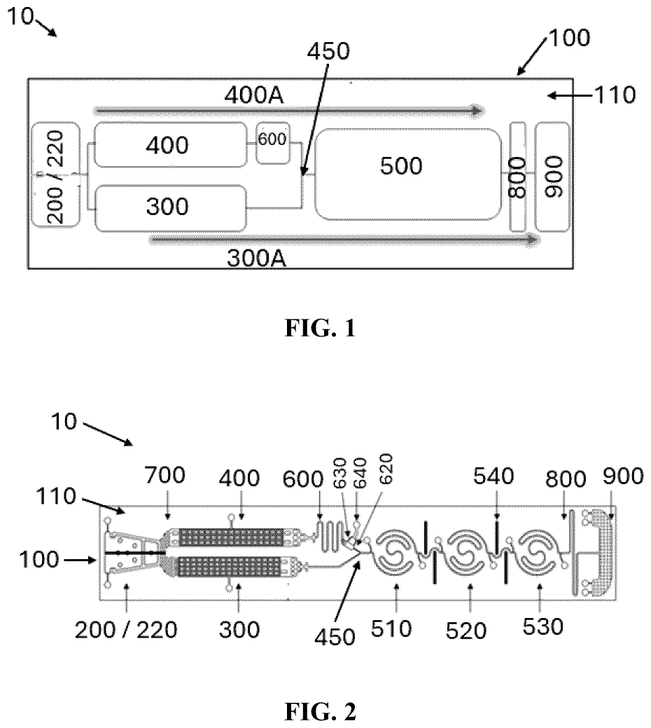

In the present disclosure, as shown in FIGS. 1-4, dried or lyophilized chemiluminescent substrate and dried or lyophilized detection antibody conjugated with an enzyme are reconstituted via contact with a liquid sample and travel along different flow paths (300A, 400A), with the reconstituted chemiluminescent substrate flow path being delayed by a first delay line included in the flow path 400A. This delay allows the antibody conjugated with an enzyme to contact immobilized capture antibodies in the one or more reaction channels 500 before contacting the chemiluminescent substrate, so that the chemiluminescent substrate can react with the conjugated enzyme upon reaching immobilized capture antibodies and antibody conjugated with the enzyme to generate the immunoassay signal. The microchannel lateral flow assay lab-on-a-chip device allows for highly sensitive detection or the target biomolecule in a simple one-step assay.

Various non-limiting embodiments of the present disclosure will now be described to provide an overall understanding of the principles of the structure, function, and use of the apparatuses, systems, methods, and processes disclosed herein. One or more examples of these non-limiting embodiments are illustrated in the accompanying drawings. Those of ordinary skill in the art will understand that systems and methods specifically described herein and illustrated in the accompanying drawings are non-limiting embodiments. The features illustrated or described in connection with one non-limiting embodiment may be combined with the features of other non-limiting embodiments. Such modifications and variations are intended to be included within the scope of the present disclosure.

Described herein is a microchannel lateral flow assay lab-on-a-chip device 10 for point-of-care testing for detecting a target in a sample. The microchannel lateral flow assay lab-on-a-chip device 10 comprises a chip 100 having a surface 110 and multiple channels formed on the surface 110 beginning with a sample loading channel 200 configured to receive the sample. The sample loading channel 200 may divide the sample into a first sample portion and a second sample portion. As shown in the schematic of FIG. 1, the channels on the surface 110 of the chip 100 form a first flow path 300A and a second flow path 400A. With further reference to FIG. 2 as well as FIG. 1, the first flow path 300A may comprise the sample loading channel 200, the detection antibody conjugate channel 300, and the one or more reaction channels 500, as well as optionally including a second delay line 800, and a waste reservoir 900. The second flow path 400A may comprise the sample loading channel 200, the chemiluminescent substrate channel 400, and the one or more reaction channels 500, as well as optionally including a second delay line 800, and a waste reservoir 900. As shown in FIGS. 1 and 2, the first sample portion and the second sample portion may flow through the first flow path 300A and the second flow path 400A, respectively, in that order.

In some embodiments, still referring to FIGS. 1-4, the microchannel lateral flow assay lab-on-a-chip device 10 comprises a sample loading channel 200 configured to receive the sample. The microchannel lateral flow assay lab-on-a-chip device 10 may further comprise a sample loading well 220 for holding the sample as it passes through the sample loading channel 200.

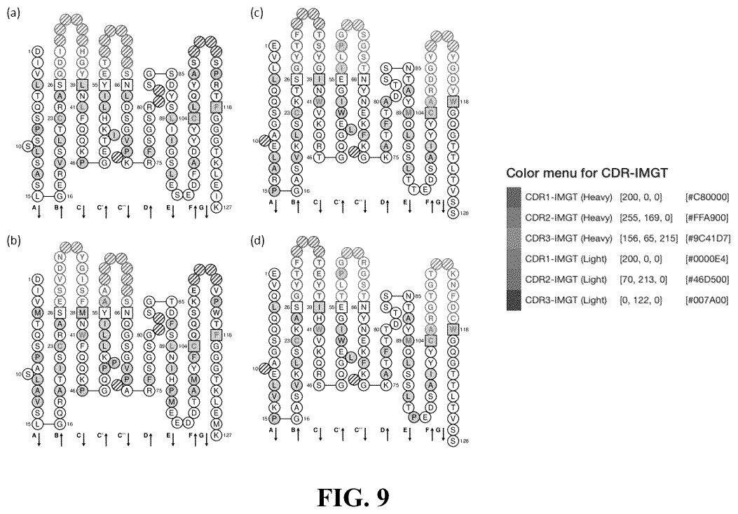

In some embodiments, still referring to FIGS. 1-4, the microchannel lateral flow assay lab-on-a-chip device 10 comprises a detection antibody conjugate channel 300. The detection antibody conjugate channel 300 may be configured to receive a first portion of the sample. The detection antibody conjugate channel 300 may receive the first portion of the sample from the sample loading channel 200, via a capillary pump 700 positioned between the sample loading channel 200 and the detection antibody conjugate channel 300, or through any other intervening channels or structures. The detection antibody conjugate channel 300 comprises dried or lyophilized detection antibody conjugated with an enzyme. The dried or lyophilized detection antibody conjugated with an enzyme may be reconstituted via contact with a liquid sample as it flows through the detection antibody conjugate channel 300. The detection antibody (DAb) is not limited and may be selected based upon the target biomolecule. For example, in some embodiments, the biomolecule may comprise proteins, antigens, or biomarkers, and the detection antibody (DAb) may comprise an immunoglobulin G (IgG) antibody, a single chain variable fragment (scFv), or other antibody formats that specifically bind the biomolecule. As a further example, as used in the Examples section of the present disclosure, the detection antibody may be a detection antibody for a protein of a virus, such as a nucleocapsid protein (N-protein) of a coronavirus such as SARS-CoV-2 virus; however, the present disclosure is not limited thereto and the detection antibody may be selected based upon its binding capacity for various target biomolecules. In some embodiments, the detection antibody may comprise a single chain variable fragment (scFv) antibody that specifically binds the target biomolecule, such as an antigen. An scFv antibody is a recombinant protein engineered from an antibody's variable regions of the very heavy (VH) and very light (VL) chains and joined by a short peptide linker. An example of a suitable scFv as the detection antibody that specifically binds nucleocapsid protein of SARS-CoV-2 virus may be referred to as scFv2 having an amino acid sequence according to SEQ ID NO: 1 or SEQ ID NO: 2. The sequences are also provided in FIG. 9 with SEQ ID NO: 1 corresponding to the (a) amino acid sequence and SEQ ID NO: 2 corresponding to the (b) amino acid sequence.

In addition, the detection antibody (DAb) is conjugated with an enzyme, such as horseradish peroxidase (HRP). The enzyme is not limited so long as it is capable of reacting, catalyzing, or otherwise interacting with the chemiluminescent substrate to generate a chemiluminescent signal. Non-limiting examples include horseradish peroxidase (HRP), alkaline phosphatase (AP), soybean peroxidase (SBP), or the like. For example, in the case of a chemiluminescent substrate comprising luminol as a chemiluminescent reagent and peroxide as a co-reagent, horseradish peroxidase may catalyze the reaction of luminol and peroxide wherein the reaction results in the emission of light.

In some embodiments, the detection antibody conjugate channel 300 may have a width of 1500 to 3500 μm, such as about 2400 μm, a height (or depth) of 100 to 500 μm, such as about 150 μm, and a volume of 1.5 to 5 μL, or about 2.75 μL.

Exemplary methods of preparing the detection antibody conjugate channel 300 having dried or lyophilized detection antibody conjugated with an enzyme are provided in the Examples section below. In some embodiments, the method of preparing the detection antibody conjugate channel 300 having dried or lyophilized detection antibody conjugated with an enzyme may comprise forming an elastomer dam structured and arranged to prevent liquid flow from the detection antibody conjugate channel to adjacent channels, adding a liquid detection antibody conjugated with an enzyme to the detection antibody conjugate channel, drying or lyophilizing the liquid detection antibody conjugated with an enzyme to form the dried or lyophilized detection antibody conjugated with an enzyme, and removing the elastomer dam. In some embodiments, the elastomer dam may be formed by an elastomeric adhesive, such as a silicone adhesive.

In some embodiments, still referring to FIGS. 1-4, the microchannel lateral flow assay lab-on-a-chip device 10 comprises a chemiluminescent substrate channel 400. The chemiluminescent substrate channel 400 may be configured to receive a second portion of the sample. The chemiluminescent substrate channel 400 may receive the second portion of the sample from the sample loading channel 200, via a capillary pump 700 positioned between the sample loading channel 200 and the detection antibody conjugate channel 300, or through any other intervening channels or structures. The chemiluminescent substrate channel 400 comprises a dried or lyophilized chemiluminescent substrate. The dried or lyophilized chemiluminescent substrate may be reconstituted via contact with a liquid sample as it flows through the chemiluminescent substrate channel 400. The dried or lyophilized chemiluminescent substrate includes a chemiluminescent reagent that is a compound capable of chemical reaction to form products along with simultaneous emission of light. In some embodiments, the chemiluminescent substrate may comprise a two-component chemiluminescent substrate wherein a chemiluminescent reagent is combined with a co-reagent to form the chemiluminescent substrate. An example of a two-component chemiluminescent substrate is luminol and peroxide. Accordingly, the chemiluminescent reagent may comprise luminol, a luminol-based compound, acridan, an acridan-based compound, a 1,2-dioxetane-based compound, or the like, and peroxide, peroxide generator, hydrogen peroxide, urea peroxide, sodium perborate, sodium percarbonate, or the like. In a two-component chemiluminescent substrate, the chemiluminescent substrate channel 400 comprises two separate channels to separate the co-reagents. For example, the chemiluminescent substrate channel 400 may comprise a first channel comprising luminol and a second channel comprising peroxide that are separately and sequentially reconstituted. In alternative embodiments, the chemiluminescent substrate may comprise a one-component chemiluminescent substrate wherein the reagents may be combined and stored in a single chemiluminescent substrate channel 400. In some embodiments, the one-component chemiluminescent substrate may comprise a mixture of a luminol derivative, an enhancer, and an oxidizing agent. In some embodiments, the enhancer may also be an electron mediator. In some embodiments, the oxidizing agent may be sodium perborate. The one-component chemiluminescent substrate may be advantageous because (a) the processing is simpler than a two-component CL that limits the possibility of non-homogenous reconstitution on the chip, and (b) the sodium perborate-based one-component CL substrate may be more stable under the variation of ambient light and humidity. It should be appreciated that many chemiluminescent substrates, in both one-component and two-component forms, are known in the field and suitable for use in the presently disclosed devices.

In some embodiments, the chemiluminescent substrate channel 400 may have a width of 1500 to 3500 μm, such as about 2100 μm, a height (or depth) of 100 to 500 μm, such as about 150 μm, and a volume of 1.5 to 5 μL, or about 2.75 μL.

Exemplary methods of preparing the chemiluminescent substrate channel 400 having dried or lyophilized chemiluminescent substrate are provided in the Examples section below. In some embodiments, the method of preparing the chemiluminescent substrate channel 400 having dried or lyophilized chemiluminescent substrate may comprise forming an elastomer dam structured and arranged to prevent liquid flow from the chemiluminescent substrate channel to adjacent channels, adding a liquid detection chemiluminescent substrate to the chemiluminescent substrate channel, drying or lyophilizing the liquid chemiluminescent substrate to form the dried or lyophilized chemiluminescent substrate, and removing the elastomer dam. In some embodiments, the elastomer dam may be formed by an elastomeric adhesive, such as a silicone adhesive.

In some embodiments, as shown in FIGS. 1-4, the detection antibody conjugate channel 300 and the chemiluminescent substrate channel 400 may be placed in parallel between the sample loading channel 200 and the one or more reaction channels 500.

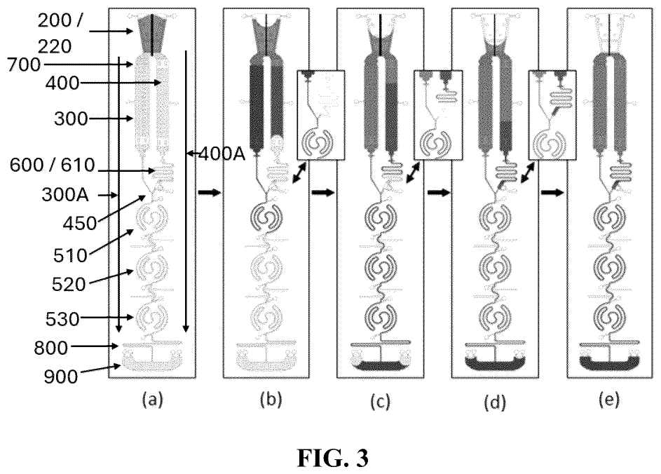

In some embodiments, as shown in FIGS. 1 and 3, the microchannel lateral flow assay lab-on-a-chip device 10 may be structured and arranged such that the first sample portion flowing through the first flow path 300A passes through the one or more reaction channels 500 (e.g., 510, 520, 530, etc.) before the second sample portion flowing through the second flow path 400A passes into the one or more reaction channels 500. The sequential flow through the first flow path 300A and the second flow path 400A where the second flow path enters the one or more reaction channels after the first flow path 300A allows for the one or more reaction channels 500 to generate a signal of immunoassay. The signal of immunoassay may include an indication of control and an indication or lack of indication of the presence of the target biomolecule in the sample. For example, FIG. 3 shows the sequential flow paths of the first flow path 300A and the second flow path 400A. For clarity, only (a) in FIG. 3 includes reference numbers. In (a), the sample is loaded in the sample loading channel 200/sample loading well 220. In (b), the sample moving through the first flow path 300A begins moving through the detection antibody conjugate channel 300 and into the first reaction channel 510, and the sample moving through the second flow path 400A begins moving through the chemiluminescent substrate channel 400. In (c), the sample portion moving through the first flow path 300A has cleared the detection antibody conjugate channel 300, filled the first reaction channel 510, the second reaction channel 520, and the third reaction channel 530, and has begun flowing through the second delay line 800 and the waste reservoir 900, whereas the sample portion moving through the second flow path 400A has begun to flow into the first delay line 600. In some embodiments, as further discussed below, the flow of the sample portion through the first delay line 600 may be reduced relative to the flow of the sample through other channels. In (d), the sample portion flowing through the first flow path 300A has cleared the first reaction channel 510, the second reaction channel 520, and the third reaction channel 530, and partially cleared the second delay line 800, whereas the sample portion flowing through the second flow path 400A is close to a merge point 450 at which a channel extending from the detection antibody conjugate channel 300 and a channel extending from the first delay line 600 merge. Finally, in (c), the sample portion flowing through the second flow path 400A flows through the first reaction channel 510, the second reaction channel 520, and the third reaction channel 530, and into the second delay line 800. In some embodiments, the unbounded antibodies may be washed by the washing buffer solution prior to the reconstituted chemiluminescent substrate flowing into the one or more reaction channels 500.

In some embodiments, the second delay line 800 may have a width of 75 to 500 μm, such as about 150 μm, a height (or depth) of 100 to 500 μm, such as about 150 μm, and a volume of 0.05 to 2 μL, or about 0.2 μL.

In some embodiments, the waste reservoir 900 may have a width of 5000 to 15000 μm, such as about 10500 μm, a height (or depth) of 100 to 500 μm, such as about 150 μm, and a volume of 0.5 to 5 μL, or about 2.2 μL.

In some embodiments, still referring to FIGS. 2-4, the microchannel lateral flow assay lab-on-a-chip device 10 may optionally further comprise vents or valves that prevent the backflow of the reconstituted detection antibody conjugated with an enzyme of the first flow path 300A at the merge point 450 into the channel extending from the first delay line 600 to the merge point 450 to contaminate the reconstituted chemiluminescent substrate that will later flow through. For example, FIG. 2 includes a backflow prevention valve 620, a hydrophobic vent 630, and a horseshoe valve 640.

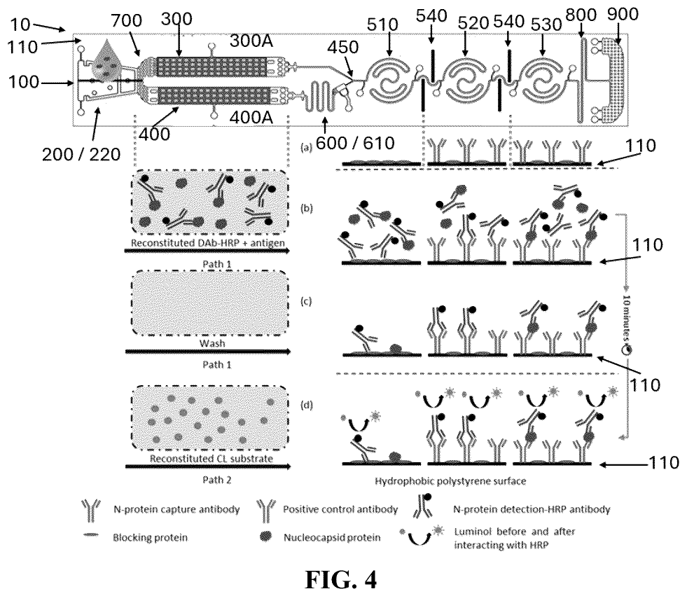

In some embodiments, still referring to FIGS. 1-4, the microchannel lateral flow assay lab-on-a-chip device 10 comprises one or more reaction channels 500. The microchannel lateral flow assay lab-on-a-chip device 10 may comprise one, two, three, or more reaction channels 500. For example, as shown in FIGS. 1-4, the microchannel lateral flow assay lab-on-a-chip device 10 may comprise a first reaction channel 510, a second reaction channel 520, and a third reaction channel 530. Each reaction channel (510, 520, 530) of the one or more reaction channels 500 may serve a different function. For example, as shown in FIG. 4, the first reaction channel 510 may serve as a negative control zone and may lack any capture antibodies that interact with the components in the sample. The second reaction channel 520 may serve as a positive control zone that includes immobilized positive control antibodies that may bind to any detection antibody conjugated with an enzyme that did not bind to the target biomolecule. The third reaction channel 530 may serve as the test zone that includes immobilized capture antibodies that bind to target biomolecule-DAb-enzyme (e.g., HRP) complex.

In embodiments, the immobilized capture antibodies (CAb) are not limited so long as they bind to the target biomolecule such that the capture antibodies may bind to the target biomolecule-DAb-enzyme (e.g., HRP) complex and form the sandwich immunocomplexes of CAb-target biomolecule-DAb-HRP. Alternatively, the immobilized capture antibodies (CAb) may be structured as a competitive assay. The immobilized capture antibodies (CAb) may comprise an immunoglobulin G (IgG) antibody, a single chain variable fragment (scFv), or other antibody formats that specifically bind the biomolecule. For example, as used in the Examples section of the present disclosure, the immobilized capture antibody may be an immobilized capture antibody for a protein of a virus, such as a nucleocapsid protein of a coronavirus such as SARS-CoV-2 virus; however, the present disclosure is not limited thereto and the immobilized capture antibody may be selected based upon its binding capacity for various target biomolecules. In some embodiments, the immobilized capture antibody may comprise a single chain variable fragment (scFv) antibody. An example of a suitable scFv as the capture antibody that specifically binds nucleocapsid protein of SARS-CoV-2 virus may be referred to as scFv2 having an amino acid sequence according to SEQ ID NO: 3 or SEQ ID NO: 4. The sequences are also provided in FIG. 9 with SEQ ID NO: 3 corresponding to the (c) amino acid sequence and SEQ ID NO: 4 corresponding to the (d) amino acid sequence.

In some embodiments, still referring to FIGS. 1-4, the one or more reaction channels 500 may have a spiral shape such that the one or more reaction channels 500 comprise a spiral microfluidic channel. The spiral reaction channels 510, 520, 530 may have higher surface-to-volume ratio when compared to a well of conventional 96-well plate. The high surface-to-volume ratio may result in higher amount of capture antibody immobilization. This may result in high optical signal output with small sample volume. The meandering structure following the reaction channels 510, 520, 530 retards the flow and allows for longer incubation in the reaction channels 510, 520, 530. Each of the spiral reaction channels 510, 520, 530 may have a width of 100 to 500 μm, such as about 150 μm, a height (or depth) of 100 to 500 μm, such as about 150 μm, and a volume of 0.5 to 4 μL, or about 0.9 μL.

In some embodiments, the microchannel lateral flow assay lab-on-a-chip device 10 comprises a first delay line 600. As mentioned above, the flow of the sample through the first delay line 600 may be reduced relative to the other components of the microchannel lateral flow assay lab-on-a-chip device 10. For example, the first delay line 600 may reduce the flow velocity of the liquid sample through the second flow path 400A relative to the flow velocity of the liquid sample through the first flow path 300A wherein the liquid sample flowing through the first flow path 300A flows through the one or more reaction channels 500 before the liquid sample flowing through the second flow path 400A. In one or more embodiments, the time for the liquid sample to flow through the second flow path 400A to reach the one or more reaction channels 500 may be at least 2 minutes longer than the time for the liquid sample to flow through the first flow path 300A, or at least 5 minutes longer, or at least 8 minutes longer, or at least 10 minutes longer. In one or more embodiments, the time for the liquid sample to flow through the second flow path 400A to reach the one or more reaction channels 500 may be 2 to 20 minutes longer than the time for the liquid sample to flow through the first flow path 300A, or 2 to 15 minutes longer, or 2 to 10 minutes longer, or 5 to 20 minutes longer, or 5 to 15 minutes longer, or 5 to 10 minutes longer, or 8 to 20 minutes longer, or 8 to 15 minutes longer, or 8 to 10 minutes longer.

In some embodiments, the first delay line 600 may have a width of 75 to 500 μm, such as about 150 μm, a height (or depth) of 100 to 500 μm, such as about 150 μm, and a volume of 0.05 to 2 μL, or about 0.2 μL.

In some embodiments, the first delay line 600 comprises a coating deposited on its surface that reduces the flow velocity of the sample through the first delay line 600. For example, the coating may be relatively hydrophobic as exhibited by an increased water contact angle. The degree of hydrophobicity and corresponding increased water contact angle may depend upon a number of factors, including the amount of flow reduction and the desired length of delay. For example, in some embodiments, the first delay line 600 may have a water contact angle of at least 40 degrees, at least 50 degrees, at least 60 degrees, at least 70 degrees, at least 75 degrees, or about 80 degrees. In some embodiments, the first delay line 600 may have a water contact angle of no more than 110 degrees, no more than 100 degrees, no more than 90 degrees, no more than 95 degrees, or no more than 85 degrees. In some embodiments, the first delay line 600 may have a water contact angle of 40 to 110 degrees, 40 to 100 degrees, 40 to 90 degrees, 40 to 95 degrees, 40 to 85 degrees, 50 to 110 degrees, 50 to 100 degrees, 50 to 95 degrees, 50 to 90 degrees, 50 to 85 degrees, 60 to 110 degrees, 60 to 100 degrees, 60 to 95 degrees, 60 to 90 degrees, 60 to 85 degrees, 70 to 110 degrees, 70 to 100 degrees, 70 to 95 degrees, 70 to 90 degrees, 70 to 85 degrees, 75 to 110 degrees, 75 to 100 degrees, 75 to 95 degrees, 75 to 90 degrees, or 75 to 85 degrees.

In some embodiments, other components of the microchannel lateral flow assay lab-on-a-chip device 10 may have their surfaces modified by deposition of a coating thereon. For example, the sample loading channel 200, detection antibody conjugate channel 300, chemiluminescent substrate channel 400, the second delay line 800, and the waste reservoir 900 may have relatively hydrophilic coatings having low water contact angles and relatively high flow velocities. For example, these components may have water contact angles of 5 to 40 degrees, 5 to 30 degrees, 5 to 25 degrees, 10 to 40 degrees, 10 to 30 degrees, 10 to 25 degrees, 15 to 40 degrees, 15 to 30 degrees, 15 to 25 degrees, 20 to 40 degrees, 20 to 30 degrees, or 20 to 25 degrees, or about 20 degrees. Likewise, the valves and vents, such as the backflow prevention valve 620, the hydrophobic vent 630, and the horseshoe valve 640, may have a more hydrophobic coating in order to repel fluid, and may have a water contact angle of greater than 85 degrees, from 85 to 120 degrees, 85 to 110 degrees, or 90 to 110 degrees, or about 100 degrees.

In some embodiments, the backflow prevention valve 620 may have a width of 25 to 150 μm, such as about 75 μm, a height (or depth) of 25 to 150 μm, such as about 75 μm, and a volume of 0.01 to 0.5 μL, or about 0.05 μL.

As discussed above, the water contact angle may depend on the hydrophilicity or hydrophobicity of the coating. Accordingly, the coating may be modified to be more or less hydrophilic or hydrophobic. Any suitable coating may be used so long as it is inert to the sample flowing through the microchannel lateral flow assay lab-on-a-chip device 10.

In addition, the chip may be treated with O₂ plasma in a plasma preen system prior to coating.

In some embodiments, the one or more reaction channels 500 may be excluded from plasma treating or coating as the naturally hydrophobic surface of the chip 100 may be beneficial for the capture antibody immobilization and blocking.

In some embodiments, as shown in FIGS. 2-4, the microchannel lateral flow assay lab-on-a-chip device 10 may comprise a capillary pump 700. The capillary pump assists in moving the sample passively. For example, the capillary pump 700 may provide a higher flow rate for washing of unbounded reagent.

In some embodiments, the capillary pump 700 may have a height (or depth) of 100 to 500 μm, such as about 150 μm, and a volume of 0.05 to 2 μL, or about 0.2 μL.

In some embodiments, the microchannel lateral flow assay lab-on-a-chip device 10 may be free of any non-passive pumps, such as an air pump, that increases the flow of the sample through the microchannel lateral flow assay lab-on-a-chip device 10.

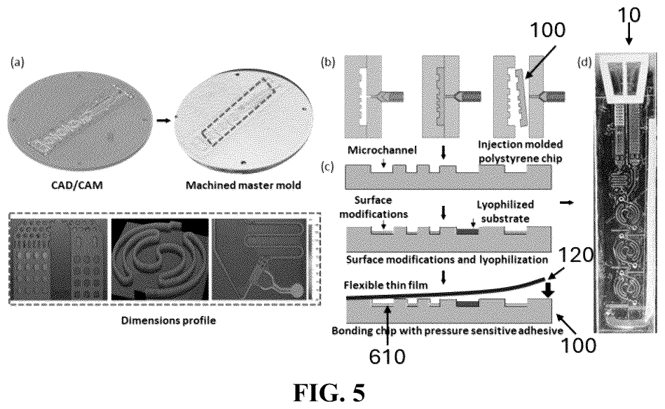

In some embodiments, as shown in FIG. 5 part (c), the microchannel lateral flow assay lab-on-a-chip device 10 further comprises a film 120 over the surface 110 of the chip 100. The film 120 may seal the channels. The film 120 may comprise any suitable material. For example, the film 120 may be a thin, flexible film capable of being adhered to the surface 110 of the chip 100. In some embodiments, the film 120 may be an adhesive tape, such as a pressure-sensitive adhesive tape. In some embodiments, the film 120 may be applied using a pneumatic press.

In some embodiments, the chip 100 may be any suitable material. For example, the chip 100 may be a polymeric material. In some embodiments, the chip 100 may be polystyrene. Polystyrene may be a suitable material because it is capable of being injection-molded, is often used in conventional 96-well plates, and is chemically inert, hydrophobic, relatively transparent, and of relatively low cost. An exemplary method of making a polystyrene chip 100 is provided in the Examples section.

In some embodiments, the volume of sample added to the microchannel lateral flow assay lab-on-a-chip device 10 sample loading channel 200 and/or sample loading well 220 may be on the microliter scale. For example, the sample may have a total volume of 1 μL to 20 μL, or from 5 μL to 15 μL. However, the microchannel lateral flow assay lab-on-a-chip device 10 may be adapted for larger or smaller sample volumes.

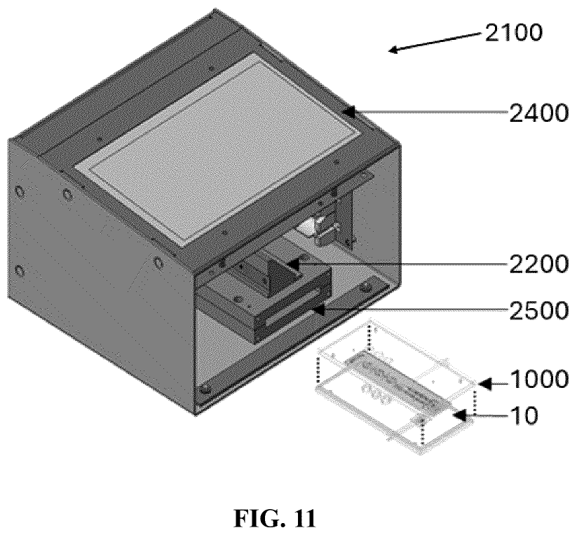

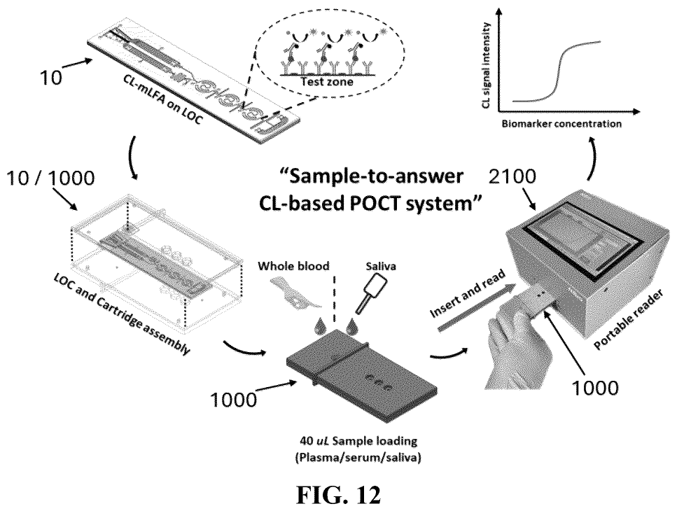

In some embodiments, referring now to FIGS. 11 and 12, the microchannel lateral flow assay lab-on-a-chip device 10 may further comprise a cartridge 1000 that houses the microchannel lateral flow assay lab-on-a-chip device 10. The cartridge may prevent ambient light from contacting the microchannel lateral flow assay lab-on-a-chip device 10 and may act as a means of interface for easy sample loading as well as equal sample splitting to each flow path. The cartridge 1000 may be fabricated using any suitable method, such as using a polymer 3D printer to accommodate the microchannel lateral flow assay lab-on-a-chip device 10. In embodiments, the microchannel lateral flow assay lab-on-a-chip device 10 may be mounted inside the cartridge 1000, and then a double-sided adhesive tape may be used between the microchannel lateral flow assay lab-on-a-chip device 10 and a sample loading port of the cartridge to prevent leakage during sample loading. The cartridge 1000 may have holes that align with the one or more reaction channels 500 such that any chemiluminescent immunoassay signal may be detected. Using distinct holes for each of the one or more reaction channels 500 may allow for less crosstalk between reaction channels 500 during analysis. The microchannel lateral flow assay lab-on-a-chip device 10 may further comprise crosstalk prevention windows 540 to limit crosstalk between the reaction channels 500.

Also disclosed is a method for detecting a target in a sample using the microchannel lateral flow assay lab-on-a-chip device 10 disclosed herein. The method may comprise loading the sample into the sample loading channel 200, allowing the first portion of the sample to flow through the detection antibody conjugate channel 300 and the one or more reaction channels 500, allowing the second portion of the sample to flow through the chemiluminescent substrate channel, the delay line, and the one or more reaction channels, wherein the first portion of the sample exits the one or more reaction channels before the second sample enters the one or more reaction channels, and identifying any immunoassay signal. In some embodiments, the second portion of the sample may have a residence time in the first delay line 600 of 5 to 20 minutes, or 5 to 15 minutes, or 5 to 10 minutes, or 8 to 20 minutes, or 8 to 15 minutes, or 8 to 10 minutes.

Also disclosed is a method for making the microchannel lateral flow assay lab-on-a-chip device of the present disclosure. The method may comprise forming the chip via injection molding, forming an elastomer dam structured and arranged to prevent liquid flow from the detection antibody conjugate channel to adjacent channels, adding a liquid detection antibody conjugated with an enzyme to the detection antibody conjugate channel, drying or lyophilizing the liquid detection antibody conjugated with an enzyme to form the dried or lyophilized detection antibody conjugated with an enzyme, and removing the elastomer dam, forming an elastomer dam structured and arranged to prevent liquid flow from the chemiluminescent substrate channel to adjacent channels, adding a liquid detection chemiluminescent substrate to the chemiluminescent substrate channel, drying or lyophilizing the liquid chemiluminescent substrate to form the dried or lyophilized chemiluminescent substrate, and removing the elastomer dam, depositing the coating to the first delay line, and depositing the film over the surface of the chip.

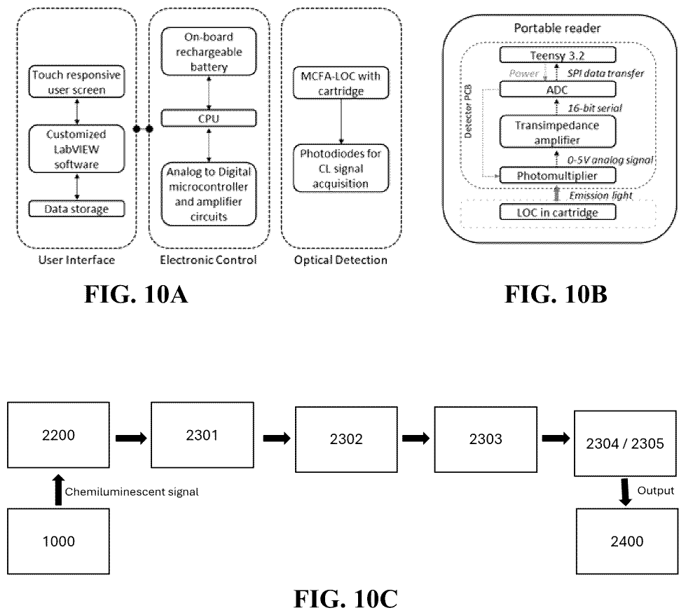

Also disclosed is a system 2000 for detecting a target in a sample. In some embodiments, referring now to FIGS. 10C and 11-12, comprising the microchannel lateral flow assay lab-on-a-chip device 10 of the present disclosure and an electronic reader structured and arranged to receive and read the microchannel lateral flow assay lab-on-a-chip. The system may receive the cartridge in a metal enclosure 2500. The electronic reader 2100 may be structured and arranged to receive the microchannel lateral flow assay lab-on-a-chip device 10 within an optical detection unit 2200. The optical detection unit may comprise photodiodes and/or photomultipliers. The electronic reader 2100 may further comprise an electronic control unit 2300. The electronic control unit 2300 may comprise a trans-impedance amplifier circuit 2301 structured and arranged to convert the current to a signal voltage and amplify the signal voltage based on feedback resistance using a potentiometer, an analog-digital converter 2302 structured and arranged to convert the signal voltage to a digital signal that is transferred to a microcontroller 2303 via a serial peripheral interface 2304, and a computer processing unit 2305 structured and arranged to receive the digital signal from the microcontroller 2303. The electronic reader 2100 may further comprise a user interface 2400 structured and arranged to provide a result derived from the digital signal. The user interface 2400 may allow the user to monitor and analyze the measured results (output) as well as control the photodiode and/or photomultiplier measurement via the computer processing unit 2305.

The terms “free” and “substantially free,” when used to describe the concentration and/or absence of a particular constituent component means that the constituent component is not intentionally added.

Ranges can be expressed herein as from “less than or equal to” one particular value, and/or to “less than or equal to” another particular value. When such a range is expressed, another embodiment includes from the one particular value and/or to the other particular value. Similarly, when values are expressed as approximations, by use of the antecedent “less than or equal to,” it will be understood that the particular value forms another embodiment. It will be further understood that the endpoints of each of the ranges are significant both in relation to the other endpoint, and independently of the other endpoint. Any ranges used herein include all ranges and subranges and any values there between unless explicitly stated otherwise.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

Directional terms as used herein—for example up, down, right, left, front, back, top, bottom—are made only with reference to the figures as drawn and are not intended to imply absolute orientation.

Unless otherwise expressly stated, it is in no way intended that any method set forth herein be construed as requiring that its steps be performed in a specific order, nor that with any apparatus specific orientations be required. Accordingly, where a method claim does not actually recite an order to be followed by its steps, or that any apparatus claim does not actually recite an order or orientation to individual components, or it is not otherwise specifically stated in the claims or description that the steps are to be limited to a specific order, or that a specific order or orientation to components of an apparatus is not recited, it is in no way intended that an order or orientation be inferred, in any respect. This holds for any possible non-express basis for interpretation, including: matters of logic with respect to arrangement of steps, operational flow, order of components, or orientation of components; plain meaning derived from grammatical organization or punctuation, and; the number or type of embodiments described in the specification.

As used herein, the singular forms “a,” “an” and “the” include plural referents unless the context clearly dictates otherwise. Thus, for example, reference to “a” component includes aspects having two or more such components, unless the context clearly indicates otherwise.

Reference throughout the specification to “various embodiments,” “some embodiments,” “one embodiment,” “some example embodiments,” “one example embodiment,” or “an embodiment” means that a particular feature, structure, or characteristic described in connection with any embodiment is included in at least one embodiment. Thus, appearances of the phrases “in various embodiments,” “in some embodiments,” “in one embodiment,” “some example embodiments,” “one example embodiment,” or “in an embodiment” in places throughout the specification are not necessarily all referring to the same embodiment. Furthermore, the particular features, structures or characteristics may be combined in any suitable manner in one or more embodiments.

In various embodiments disclosed herein, a single component can be replaced by multiple components and multiple components can be replaced by a single component to perform a given function or functions. Except where such substitution would not be operative, such substitution is within the intended scope of the embodiments.

The examples discussed herein are examples only and are provided to assist in the explanation of the apparatuses, devices, systems and methods described herein. None of the features or components shown in the drawings or discussed below should be taken as mandatory for any specific implementation of any of these the apparatuses, devices, systems or methods unless specifically designated as mandatory. For ease of reading and clarity, certain components, modules, or methods may be described solely in connection with a specific figure. Any failure to specifically describe a combination or sub-combination of components should not be understood as an indication that any combination or sub-combination is not possible. Also, for any methods described, regardless of whether the method is described in conjunction with a flow diagram, it should be understood that unless otherwise specified or required by context, any explicit or implicit ordering of steps performed in the execution of a method does not imply that those steps must be performed in the order presented but instead may be performed in a different order or in parallel.

Having shown and described various versions in the present disclosure, further adaptations of the methods and systems described herein may be accomplished by appropriate modifications by one of ordinary skill in the art without departing from the scope of the present disclosure. Several of such potential modifications have been mentioned, and others will be apparent to those skilled in the art. For instance, the examples, versions, geometrics, materials, dimensions, ratios, steps, and the like discussed above are illustrative and are not required. Accordingly, the scope of the present disclosure should be considered in terms of the following claims and is understood not to be limited to the details of structure and operation shown and described in the specification and drawings.

### Examples

The various embodiments of systems and processes of the present disclosure will be further clarified by the following examples. The examples are illustrative in nature, and should not be understood to limit the subject matter of the present disclosure.

### Microfabrication of Master Mold and Injection-Molded Polymer Chips:

The lab-on-chip (LOC) for chemiluminescent microchannel lateral flow assay (CL-mLFA) was injection-molded with polystyrene and sealed with adhesive tape using a pneumatic press for low-cost fabrication. An insertable aluminium disc was used for injection molding. Thus, as shown in FIG. 5 part (a), a disc-mold of the polymer chip was first designed using CAD/CAM software, Mastercam 2023 (Mastercam®, USA), according to the dimensions defined in Table 1 below. Machining a circular aluminium disc-mold was carried out using a 5-axis milling machine Microlution 5100S (Microlution Inc., USA). The micro-machined aluminium disc-mold with a diameter of 3 inches is also shown in FIG. 5 part (a).

TABLE 1

| | Width | Height | Volume |
|---|---|---|---|
| Microfluidics elements on LOC | (μm) | (μm) | (uL) |
| Capillary pump | N/A | 150 | 0.22 |
| Detection antibody conjugate channel | 2400 | 150 | 2.75 |
| Chemiluminescent substrate Channel | 2100 | 150 | 2.75 |
| First delay line | 150 | 150 | 0.18 |
| Backflow prevention valve | 75 | 75 | 0.05 |
| Reaction channel(s) | 150 | 150 | 0.86 |
| Second delay line | 150 | 150 | 0.5 |
| Waste reservoir | 10500 | 150 | 2.17 |

After completing the fabrication, the dimensions of the micro-machined disc-mold were inspected using a contour microscope (Bruker, USA), as shown in FIG. 5 part (a) (lower panel). Then, the disc-mold was inserted in the injection molding machine BOY22 (BOY Machines Inc., Germany) and chips were injection-molded using polystyrene, 3100 (INEOS Styrolution, Germany), as illustrated in FIG. 5 part (b). The injection-molded polystyrene chips were processed by surface modification and lyophilization (as further described herein below) on the open chips, then sealed using a pressure-sensitive adhesive tape, as shown in FIG. 5 part (c). A picture of a fabricated LOC for CL-based microchannel lateral flow assay (CL-mLFA) is shown in FIG. 5 part (d).

### CL-mLFA Assay Preparation on Polymer LOC:

The following reagents were procured to perform the N-protein immunoassay of the SARS-CoV-2 virus: Rabbit capture antibody from Sino Biological, USA (68097-R150); detection antibody from Sino Biological, USA (40588-RC02) and phosphate-buffered saline (PBS) as CAb coating buffer and washing buffer from Abcam, USA (ab285410). The blocking buffer was used for blocking the reaction sites and as a diluent buffer from Thermofisher, USA (StartingBlock Blocking Buffer™, 37578). Chemifluorescent substrate was from Thermofisher, USA (QuantaRed, 15159). One component chemiluminescent substrate based on sodium perborate was from Cyanagen, Italy (Westar-one extreme, XLSU180). Artificial serum (AS) was from CST Technologies Inc., USA (Serasub™). HRP conjugation kit was from Abcam, USA (Lightning-Link®, ab102890), and the SARS-CoV-2 pseudovirus was from Virongy, USA (Ha-CoV-2). Specifically, the scFv antibodies utilized for the N-protein assay were produced by Dr. Keun Seo's lab at Mississippi State University, who custom-developed scFv antibodies specific to the N-protein.

The assay was prepared by cutting and drilling the vent holes on the injection-molded polystyrene chips, cleaning with isopropyl alcohol, deionized water, and drying with nitrogen gas. To modify the surface of microchannels with a desired hydrophilicity or hydrophobicity, all channels and chambers of the chips were treated with O₂ plasma (30 seconds, 1 SCFH, 100% power level) in the plasma-preen system (Plasmatic Systems Inc., USA), except the spiral reaction channels, which were covered with adhesive tape, because they should have a hydrophobic surface for the CAb immobilization and blocking step. Then, the surface of the microchannel was modified to realize the sequential dual flows of the two flow paths on the LOC via coatings. Surface modification coating solutions (X100 and A30, Joninn ApS, Denmark) were used to attain the desired hydrophilic and hydrophobic surfaces, as summarized in Table 2, below. Next, the reaction zones were prepared for the CAb immobilization using the following protocol. On the test reaction zone, the CAb was in the desired concentration, diluted in a coating buffer, incubated for 20 minutes, and washed with PBS. Then, the StartingBlock blocking buffer was incubated for 20 minutes and washed with PBS. For the negative control reaction zone, the CAb incubation step was omitted, so only blocking proteins existed on the reaction zone surface. Once the reaction zones were incubated with the desired immunoassay reagents, they were vacuum-dried inside a vacuum desiccator (SP Bel-Art, USA) at −0.1 MPa for 30 minutes.

TABLE 2

| | Coating | Contact |
|---|---|---|
| Microfluidic | condition | angle |
| element | (% V) | (degrees) |
| Sample loading channel | | |
| Detection antibody | 100% X100 | ~20 |
| conjugate channel | | |
| Chemiluminescent | | |
| substrate channel | | |
| First Delay Line | 30% X100 + 70% A30 | ~80 |
| Horseshoe valve | 100% A30 | ~100 |
| First reaction channel | | |
| Second reaction channel | Assay | ~13 |
| Third reaction channel | | |
| Second Delay Line | 100% X100 | ~20 |

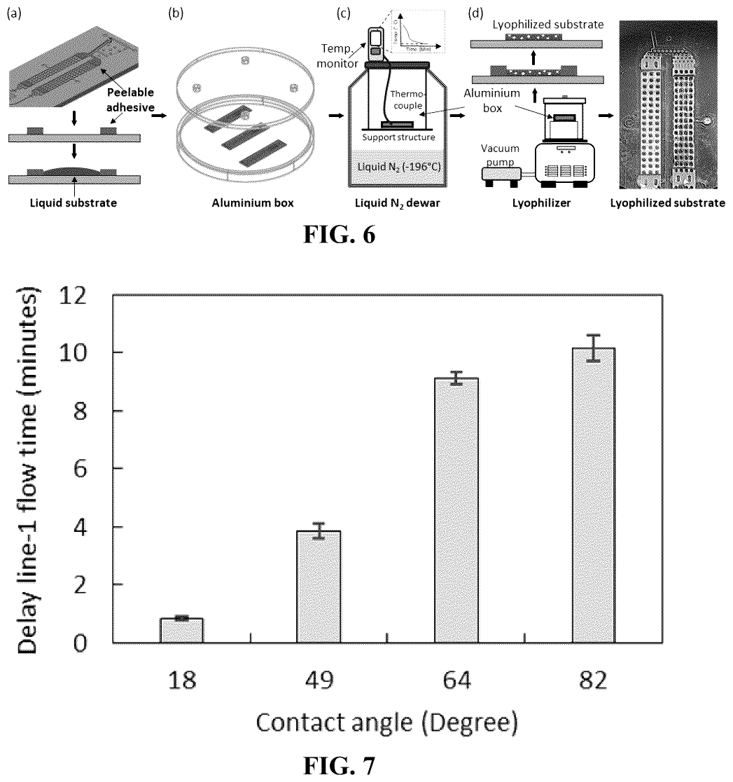

Lyophilization was performed by forming flexible elastomer dams that can be easily prepared and removed after the lyophilization process at opposing ends of the channels so that the liquid can be held in the channel constructed between the dams. To perform the lyophilization process of DAb-HRP and CL substrate, a rapid freezing step of these two liquids was required ahead of the drying step inside a liquid nitrogen dewar. A peelable, quick-dry silicone adhesive from World Precision Instruments, USA (Kwik-cast low toxicity silicone sealant), that is not harmful to polystyrene was used to create dam structures as shown in FIG. 6 part (a). After lyophilization, the silicone adhesive dams were peeled off easily, and the areas still maintained hydrophilicity after the removal of the dams. A 3 μL of DAb-HRP and a 3 μL of one component CL substrate were dispensed, and the polymer chips containing liquid reagents were put in a custom-designed aluminium box, as shown in FIG. 6 part (b). An aluminium box of 101.6 mm in diameter and 6.35 mm in thickness was used for achieving a stable temperature equilibrium during the freezing step and holding the temperature stability at the beginning of the drying step in the lyophilizer. The aluminium box was first located inside a liquid nitrogen dewar for stable freezing, such that the box was positioned at around 127 mm from the liquid nitrogen surface. The temperature during the entire freezing step was monitored through a thermocouple attached to the inside of the aluminium box, as shown in FIG. 6 part (c). Once the temperature reached −160° C., the aluminium box was immediately transferred onto the lyophilizer rack (FreeZone 1 Liter −54° C. benchtop freeze dryer, Labconco, USA) with the collector preset at −54° C. and 0.010 mBar of vacuum was applied for 10 hours. As shown in FIG. 6 part (d), the substrate after lyophilization had a porous structure, that may assist with easy and quick reconstitution. The chips prepared with lyophilized reagents were bonded with a pressure-sensitive adhesive tape IST-121 QuickSeal, 60 μm in thickness (IST Scientific, UK), using a pneumatic press set to 60 psi for 30 seconds as shown in FIG. 5 part (c), and FIG. 5 part (d) shows the fully prepared LOC for CL-mLFA.

### Delay Time Control on CL-mLFA LOC:

Prior chemiluminescent-based microchannel lateral flow assay lab-on-a-chip device based on vent opening for triggering the flow of the chemiluminescent substrate, which was implemented manually once the flow of the detection antibody was complete. To eliminate this user intervention, the present chemiluminescent-based microchannel lateral flow assay lab-on-a-chip device incorporated an automatic delay by coating the first delay line to have a delay of around 10 minutes so that the sample from the second flow path could merge autonomously after the desired delay without any user interventions, such as opening another vent. FIG. 7 depicts the correlation between the water contact angle of the coating and delay time. Based on this result, a contact angle of about 80°, giving the desired 10 minutes of delay time, was chosen. Table 3 below shows how the appropriate delay was achieved by mixing a hydrophilic coating (X100) and a hydrophobic coating (A30) at different ratios to get a range of delay line filling times and contact angles. From Table 3, a mixture containing 30% of X100 (volume %) and 70% of A30 (volume %) was used to provide the desired delay time of around 10 minutes.

TABLE 3

| Hydrophilic | Hydrophobic | Delay line-1 fill | |
|---|---|---|---|
| X100 (V %) | A30 (V %) | timing (minutes) | Contact angle (degree) |
| 100 | 0 | <1 | 18 |
| 50 | 50 | 3-4 | 49 |
| 40 | 60 | 7-8 | 64 |
| 30 | 70 | 9-11 | 82 |
| 0 | 100 | >30 | 103 |

### Cartridge Assembly:

The cartridge for the CL-mLFA LOC was fabricated using a polymer 3D printer (UltiMaker, USA). In detail, the CL-mLFA LOC was mounted inside the cartridge, and then a double-sided adhesive tape of 1 mm thick was used between the CL-mLFA LOC and the sample loading port of the cartridge to prevent leakage during sample loading, as shown in FIG. 12 (LOC and cartridge assembly). Thus, the sample loading port allowed the loaded sample to split and flow to each path on the CL-mLFA LOC. Thus, only when the cartridge was fully assembled with the CL-mLFA LOC was the cartridge ready to perform the CL-mLFA. The cartridge had three opening holes of 6 mm in diameter that aligned with the three spiral reaction channels on the CL-mLFA LOC, thus the CL signals were able to reach the photodiodes and prevent the crosstalk between the spiral reaction channels.

### Characterization of Lyophilized One-Component CL Substrate:

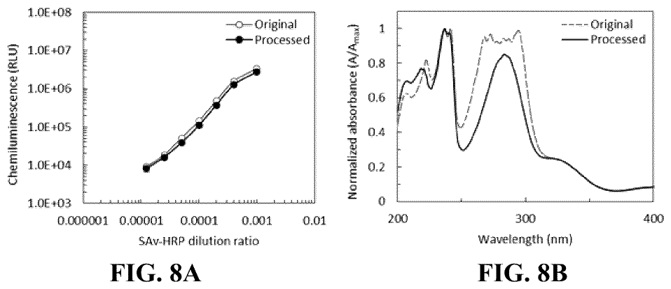

Experiments were conducted to confirm that the one-component CL substrate was compatible with the lyophilization process. The lyophilization process, performed as described above, was followed by comparison of the intensity of the CL substrate before and after lyophilization, with results shown in FIG. 8A. Specifically, FIG. 8A shows a comparison of one component CL intensity before (original) and after lyophilization (processed) with different dilutions of SAv-HRP coated on Optimiser™ microplate. Each data point is the mean of triplicate and error bar represents the standard deviation, but each error bar is smaller than the data point. Here, Optimiser™ microchannel-based microplate (MiCo BioMed, South Korea) coated with Streptavidin-HRP (Sav-HRP) at different concentrations was used to measure CL signal intensity for both the fresh CL substrate in liquid form and the lyophilized and reconstituted CL substrate using a BioTek reader (Synergy HT, Agilent, USA). The sodium perborate-based, one-component CL substrate showed a matched trend along a wide range of Sav-HRP concentrations on a reaction surface, confirming its suitability for the lyophilization process and CL-mLFA on the LOC. To investigate further the chemical nature of the CL substrate during processing, we conducted ultraviolet-visible (UV-Vis) absorption spectroscopy using NanoDrop™ Onee spectrophotometer (ThermoFisher, USA). FIG. 8B shows the UV-Vis absorbance spectrum measured using Nanodrop™ One of (i) the control (original), where the CL substrate was used in its liquid form as a standard, and (ii) the substrate solution obtained from the reconstitution of the lyophilized CL substrate. The recovery rate of the CL substrate was found to be over 85%, which indicated the one-component CL substrate used in this work was acceptable for performing the CL-based mLFA on LOC after the lyophilization process.

### Development of a Custom-Designed Portable Reader for POCT:

A custom-designed portable reader was developed to measure the CL signal from the CL-mLFA on LOC assembled in the cartridge. The cartridge helped to align the reaction zones on the LOC with the photodiodes in the portable reader, allowing the emitted CL signal to efficiently reach the respective photodiodes, preventing crosstalk between the photodiodes and avoiding ambient light as well. The reader has an outer dimension of 188×170×114 mm and houses optical photodiodes as part of the detector circuit, a microcontroller, a touch screen display to monitor the CL signals using a software application built with LabVIEW, and an enclosure to prevent ambient light. It should be appreciated that other dimensions for the portable reader are contemplated and possible.

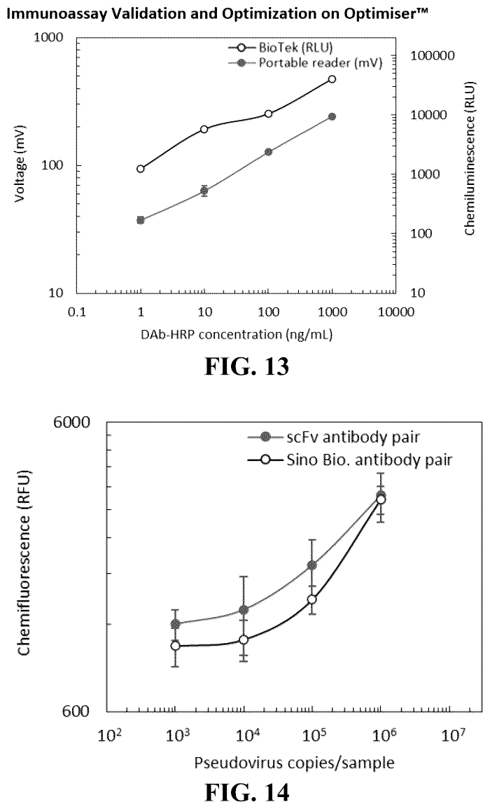

FIG. 10A shows the block diagram of the opto-electronic system that has been categorized into three subunits: the user interface, electronic control, and optical detection. The detection started with just a single click, and the CL signal measurement began. Photodiodes (MicroFC-30035-SMT-C1, onsemi, USA) have a peak detection wavelength of 420 nm that is very close to the CL peak emission wavelength of 425 nm, acquire the optical signal emitted from the reaction zones to produce a current in the micro-ampere scale (i.e., current). The current is fed to an amplifier circuit built using Op-Amp (OPA320, Texas Instruments, USA), and the trans-impedance amplifier converts this current to voltage and amplifies the signal based on the feedback resistance that can be controlled using a potentiometer. In this case, a feedback resistance of 100 KΩ showed a good performance in differentiating the signals for a given range of DAb-HRP (1 ng/ml to 1,000 ng/mL) present in the reaction zones. The analog signal is then fed to the analog-digital converter (ADS1118, Texas Instruments, USA) that converts the voltage signal to a 16-bit digital signal, which is transferred from ADS1118 to a microcontroller (Teensy 3.2, PJRC, USA) via a serial peripheral interface (SPI). Finally, the data from the microcontroller is transferred to the CPU (Intel® compute stick, Intel, USA) on request command via the serial interface. All the operations in the portable reader were controlled using a custom-developed LabVIEW software that includes the user interface for monitoring and analyzing the measured results as well as controlling the photodiode measurement via the CPU Intel Compute Stick. FIG. 10B shows the detailed flow of the opto-electronic signal in the portable reader. The setup to provide alignment of the LOC cartridge with the enclosure to prevent crosstalk, the photodiodes and the photodiode detector circuit underneath the enclosure for CL signal detection is shown in FIG. 11. To validate the functioning of the portable reader, different concentrations of DAb-HRP was incubated in the reaction zones of the LOC, and a fresh CL substrate was introduced. Multiple sets of chips were measured using the portable reader and compared with the result from a BioTek reader. In both cases, the signal showed a similar trend in a given range of DAb-HRP, as shown in FIG. 13, indicating reliability in measuring the assay performed on LOC with the portable reader. Each data point was the mean of triplicate and the error bar represented the standard deviation.

### Results and Discussion:

For diagnosing SARS-CoV-2 viral infection, a nasopharyngeal swab is commonly used as a sample. After the sample collection, the swab is inserted in a tube containing an extraction buffer, which causes the viral cell to lyse, and the proteins present inside the virus are exposed and can be used for diagnosing. The compatibility test of the extraction buffer was performed with SARS-CoV-2 pseudovirus, expressing all the proteins present in the actual virus due to its contagion and facility requirement for handling it. Since pseudoviruses are non-replicative, they are allowed to be handled in Biosafety Level 2 (BSL-2) facility.

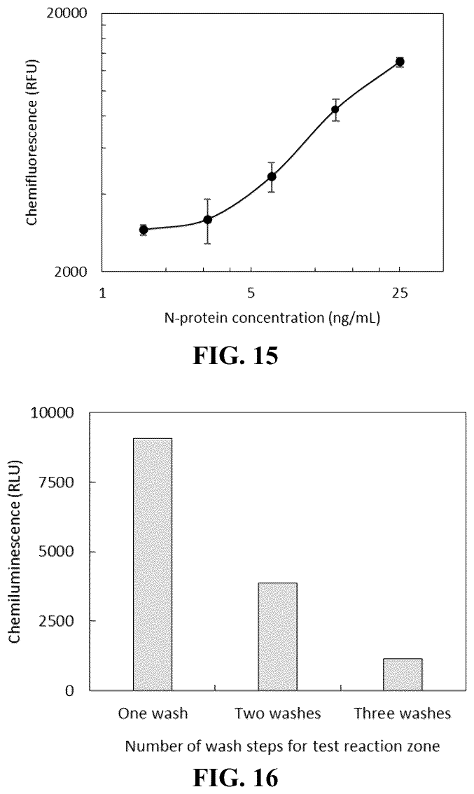

The immunoassay reagents were validated on a commercially available Optimiser™ microchannel-based microplate, which usually provides assay performance and sensitivity similar to the conventional 96-well assay by following the assay protocol based on the Optimiser™ Assay Transfer Guide. A standard curve was first obtained using the lysed sample in the extraction buffer described in the previous section to validate the assay reagents and assay protocol, including the extraction protocol. FIG. 14 shows the standard curves obtained from both the developed scFv antibody pair and commercially available antibody pair on the Optimiser™ platform for detecting N-protein, with a sample range from 10³ pseudovirus copies/sample to 10⁶ pseudovirus copies/sample. Each data point is the mean of triplicate, and the error bar represents the standard deviation. A LoD of 10³ pseudovirus copies/sample was observed for the scFv antibody pair. The obtained results were compared with the N-protein-specific IgG antibody pair from Sino Biological and the LoD was found to be 10⁵ pseudovirus copies/sample. The scFv antibody pair showed a performance of 100 times better than the commercial Sino Biological antibody pair. The assay was also performed using a purified protein spiked sample with the same assay reagents and protocols. The standard curve obtained for the purified protein sample on the Optimiser™ platform is shown in FIG. 15 with each data point being the mean of triplicate and the error bar representing the standard deviation. A detection range of 1.6 ng/mL to 100 ng/ml was tested, and it was observed that concentrations above 25 ng/ml showed saturation. An LoD of 1.6 ng/ml was achieved on this platform for the purified protein spiked sample.

### SARS-CoV-2 N-Protein Detection on LOC:

As per the assay protocol in Optimiser™ Assay Transfer Guide, at least three wash steps are required to wash away unbound molecules to reduce background noise. Keeping this as a reference, the flow protocol for CL-mLFA was as follows: A 40 μL aliquot of the sample to be tested was loaded onto the sample port of the cartridge. Due to the design of the cartridge, the sample naturally split into two equal parts of 20 μL, each following separate paths through the LOC channels. The total volume of the three spiral reaction channels and the DAb-HRP channel was approximately 2.5 μL each. The reconstituted DAb-HRP, with the loaded sample, first passed through the spiral reaction channels and started reacting there to form the sandwich immunocomplex (CAb-N-protein-DAb-HRP). The next 12.5 μL of the sample loaded initially passed through the reaction zones, which worked as a washing buffer to remove the unbound molecules from the reaction zones. The remaining 5 μL was retained in the DAb-HRP chamber and reaction zones. Then, the first flow path stopped as there was no sample remaining in the sample loading channel of the first flow path, and the fluid front did not have enough capillary pressure to pull the sample. FIG. 16 shows the signal from the test reaction channel when there was no antigen present in the sample with one, two and three washing steps. From this result, it can be clearly seen that at least three washing steps were required to remove a significant amount of the unbound DAb-HRP from the reaction channel since the signal obtained after three washes was close to the signal from a blank reaction channel. Thus, while performing an assay on LOC, enough washing steps through the microchannel were expected.

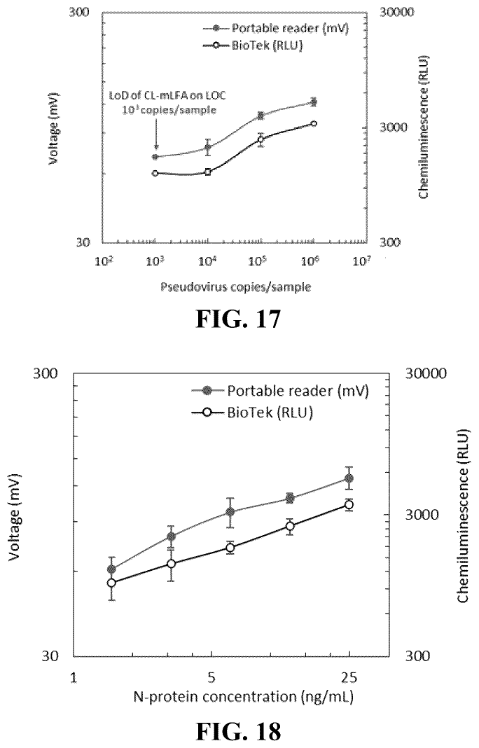

After the validation of assay reagent functionality and assay protocol on the Optimiser™ platform and optimizing the assay performance, which included the washing steps, the CL-mLFA on LOC for N-protein extracted from the lysed sample of pseudovirus was performed and analyzed with both the custom-designed portable reader and the BioTek reader. The performed assay results are depicted in FIG. 17 with each data point being the mean of triplicate and the error bar representing the standard deviation, showing that the trend was comparable for both readers, and hence, the functionality of the custom-designed portable reader was validated well. Finally, the CL-mLFA for the pseudovirus of SARS-CoV-2, which was performed on the LOC platform with the protocol developed in this work, achieved a minimum detection of 10³ copies/sample.

For a parallel comparison for various platforms using the same lysed pseudovirus test sample, we used commercially available rapid diagnostic test (RDT) kits (Flowflex® COVID-19 Antigen Home Test, USA) for COVID-19, following the kit's instruction manual. The commercially available RDTs could not show a positive result for the pseudovirus copies below 10⁵ copies/sample. On the other hand, the CL-mLFA showed a LoD of 10³ copies/sample, which achieved 100 times lower LoD than the RDTs. This result clearly showed a high potential of the CL-mLFA on the LOC platform as a next-generation POCT. The CL-mLFA for the purified protein spiked sample on the LOC platform was tested and was read using both the portable reader and the BioTek reader. The results are shown in FIG. 18 with each data point being the mean of triplicate, the error bar representing the standard deviation, and an LoD measured as 1.6 ng/ml. The achieved assay performances were similar to those of the Optimiser™ platform with a LoD of 1.6 ng/ml. This result is another confirmation of the reliable assay performance achievable with the CL-mLFA on the LOC platform.

In this work, a new CL-mLFA on the LOC platform for the sample-to-answer POCT was successfully developed and characterized for the detection of SARS-CoV-2. The CL-mLFA on LOC was composed of an on-chip lyophilized one-component CL substrate based on sodium perborate, a sequential dual flow polymer LOC, and a portable reader for POCT applications. The developed platform could perform a high-sensitive and rapid immunoassay through the sequential dual flow control of assay reagents with minimal user intervention. The lyophilization process of the one-component CL substrate was developed and characterized well, confirming its functionality after reconstitution. The long-term stability of the lyophilized CL substrate needs to be further explored for the POCT applications. CL-mLFA of the N-protein assay with both pseudovirus and purified protein samples of SARS-CoV-2 were successfully performed on the LOC platform in response to the recent pandemic, achieving a limit of detection (LoD) of 10³ copies/sample and 1.6 ng/mL, respectively, using the scFv antibody pair developed in this work. Unlike commercially available antibodies, the scFv antibodies offered high specificity to the N-protein of SARS-CoV-2, which improved the performance of the N-protein assay. The CL-mLFA on the LOC platform developed in this work is not only applicable for diagnosing infectious diseases but also other acute and chronic diseases.

It is noted that any two quantitative values assigned to a property may constitute a range of that property, and all combinations of ranges formed from all stated quantitative values of a given property are contemplated in this disclosure.

It is noted that one or more of the following claims utilize the term “where” as a transitional phrase. For the purposes of defining the present technology, it is noted that this term is introduced in the claims as an open-ended transitional phrase that is used to introduce a recitation of a series of characteristics of the structure and should be interpreted in like manner as the more commonly used open-ended preamble term “comprising.”

Having described the subject matter of the present disclosure in detail and by reference to specific aspects, it is noted that the various details of such aspects should not be taken to imply that these details are essential components of the aspects. Rather, the claims appended hereto should be taken as the sole representation of the breadth of the present disclosure and the corresponding scope of the various aspects described in this disclosure. Further, it will be apparent that modifications and variations are possible without departing from the scope of the appended claims.

What is claimed is:

1. A microchannel lateral flow assay lab-on-a-chip device for point-of-care testing for detecting a target in a sample, the chip having a surface comprising:
a sample loading channel configured to receive the sample;
a detection antibody conjugate channel comprising a dried or lyophilized detection antibody conjugated with an enzyme, the detection antibody conjugate channel configured to receive a first portion of the sample;
a chemiluminescent substrate channel comprising a dried or lyophilized chemiluminescent substrate, wherein the chemiluminescent substrate channel is configured to receive a second portion of the sample;
one or more reaction channels connected to the detection antibody conjugate channel and the chemiluminescent substrate channel, wherein at least one of the reaction channels generates a signal of immunoassay, and the at least one reaction channel that generates a signal of immunoassay comprises immobilized capture antibodies;
a first delay line between the chemiluminescent substrate channel and the one or more reaction channels, the first delay line comprising a coating;
a capillary pump; and
a film over the surface of the chip.

2. The microchannel lateral flow assay lab-on-a-chip device of claim 1, wherein the dried or lyophilized chemiluminescent substrate comprises a one-component chemiluminescent substrate.

3. The microchannel lateral flow assay lab-on-a-chip device of claim 2, wherein the one-component chemiluminescent substrate comprises mixture of a luminol derivative, an enhancer, and an oxidizing agent.

4. The microchannel lateral flow assay lab-on-a-chip device of claim 3, wherein the oxidizing agent comprises sodium perborate.

5. The microchannel lateral flow assay lab-on-a-chip device of claim 1, wherein the first delay line exhibits a water contact angle of 60 to 95 degrees.

6. The microchannel lateral flow assay lab-on-a-chip device of claim 1, wherein each of the one or more reaction channels comprises a spiral microfluidic channel.

7. The microchannel lateral flow assay lab-on-a-chip device of claim 1, wherein the chip is a polymer chip.

8. The microchannel lateral flow assay lab-on-a-chip device of claim 7, wherein the polymer chip comprises polystyrene.

9. The microchannel lateral flow assay lab-on-a-chip device of claim 1, further comprising a second delay line configured to receive the sample from the one or more reaction channels, and a waste reservoir structured and arranged to receive the sample from the second delay line.

10. The microchannel lateral flow assay lab-on-a-chip device of claim 1, wherein the first portion of the sample exits the one or more reaction channels before the second sample enters the one or more reaction channels.

11. The microchannel lateral flow assay lab-on-a-chip device of claim 1, wherein the sample has a total volume of 5 to 15 microliters.

12. The microchannel lateral flow assay lab-on-a-chip device of claim 1, wherein the target is a protein of a virus, and the detection antibody and the immobilized capture antibodies each comprise a single chain variable fragment that specifically binds the protein.

13. The microchannel lateral flow assay lab-on-a-chip device of claim 12, wherein the protein is a nucleocapsid protein and the virus is a coronavirus.

14. The microchannel lateral flow assay lab-on-a-chip device of claim 12, wherein the coronavirus is SARS-CoV-2 virus.

15. The microchannel lateral flow assay lab-on-a-chip device of claim 1, wherein the target comprises a biomolecule selected from the group consisting of proteins, hormones, antigens, and biomarkers, and the detection antibody and the immobilized capture antibodies comprise an immunoglobulin G (IgG) antibody, a single chain variable fragment (scFv), or other antibody formats that specifically bind the biomolecule, and wherein the immunoassay format is a sandwich or competitive assay.

16. The microchannel lateral flow assay lab-on-a-chip device of claim 1, wherein:
the sample loading channel comprises a coating wherein the first delay line exhibits a water contact angle of 5 to 40 degrees;
the detection antibody conjugate channel comprises a coating wherein the first delay line exhibits a water contact angle of 5 to 40 degrees; and
the chemiluminescent substrate channel comprises a coating wherein the first delay line exhibits a water contact angle of 5 to 40 degrees.

17. The microchannel lateral flow assay lab-on-a-chip device of claim 1, further comprising a cartridge that houses the microchannel lateral flow assay lab-on-a-chip.

18. A method for detecting a target in a sample using the microchannel lateral flow assay lab-on-a-chip device of claim 1, wherein the method comprises:
loading the sample into the sample loading channel;
allowing the first portion of the sample to flow through the detection antibody conjugate channel and the one or more reaction channels;
allowing the second portion of the sample to flow through the chemiluminescent substrate channel, the delay line, and the one or more reaction channels;
wherein the first portion of the sample exits the one or more reaction channels before the second sample enters the one or more reaction channels; and
identifying any immunoassay signal.

19. The method of claim 18, wherein the second portion of the sample has a residence time in the delay line of 5 to 15 minutes.

20. A method for making the microchannel lateral flow assay lab-on-a-chip device of claim 1, the method comprising:
forming the chip via injection molding;
forming an elastomer dam structured and arranged to prevent liquid flow from the detection antibody conjugate channel to adjacent channels, adding a liquid detection antibody conjugated with an enzyme to the detection antibody conjugate channel, drying or lyophilizing the liquid detection antibody conjugated with an enzyme to form the dried or lyophilized detection antibody conjugated with an enzyme, and removing the elastomer dam;
forming an elastomer dam structured and arranged to prevent liquid flow from the chemiluminescent substrate channel to adjacent channels, adding a liquid detection chemiluminescent substrate to the chemiluminescent substrate channel, drying or lyophilizing the liquid chemiluminescent substrate to form the dried or lyophilized chemiluminescent substrate, and removing the elastomer dam;
depositing the coating to the first delay line; and
depositing the film over the surface of the chip.

21. A system for detecting a target in a sample comprising:
the microchannel lateral flow assay lab-on-a-chip device of claim 1, wherein the signal of immunoassay is a chemiluminescent signal; and
an electronic reader structured and arranged to receive and read the microchannel lateral flow assay lab-on-a-chip, the electronic reader comprising:
an optical detection unit structured and arranged to read the optical signal of the chemiluminescent signal and produce a current corresponding to such reading;
an electronic control unit comprising:
a trans-impedance amplifier circuit structured and arranged to convert the current to a signal voltage and amplify the signal voltage based on feedback resistance using a potentiometer;
an analog-digital converter structured and arranged to convert the signal voltage to a digital signal that is transferred to a microcontroller via a serial peripheral interface; and
a computer processing unit structured and arranged to receive the digital signal from the microcontroller, and
a user interface structured and arranged to provide a result derived from the digital signal.
